(19) United States
(12) Patent Application Publication
(10) Pub. No.: US 2008/0177081 A1
(43) Pub. Date: Jul. 24, 2008

(54) Process for Preparation of Anastrozole

(75) Inventors: Hui-Yen Hsieh, Taoyuan (TW); Ching-Peng Wei, Taoyuan (TW)

Correspondence Address: WPAT, PC;INTELLECTUAL PROPERTY ATTORNEYS, 2030 MAIN STREET, SUITE 1300, IRVINE, CA, 92614, US

(73) Assignee: FORMOSA LABORATORIES, INC., Taoyuan (TW)

(21) Appl. No.: 11/625,226

(22) Filed: Jan. 19, 2007

(51) Int. Cl. C07D 249/08

(52) U.S. Cl. 548/262.2

(57) ABSTRACT

The present invention provides an improved process for preparing Anastrozole.

### FIELD OF THE INVENTION

The present invention relates to an improved process for the preparation of Anastrozole.

### BACKGROUND OF THE INVENTION

Anastrozole is one of the (substituted-aralkyl)-heterocyclic compounds, also known as alpha.,.alpha.,.alpha.′,.alpha.′-tetramethyl-5-(1H-1,2,4-triazol-ylmethyl)-1,3-benzenedi acetonitrile or 2,2′-[5-(1H-1,2,4-triazol-1-ylmethyl)-1,3-phenylene]di(2-methylpropionitr-ile), is represented as follows:

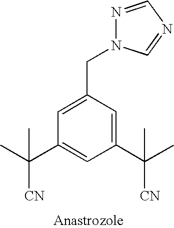

Anastrozole is a potent and selective non-steroidal aromatase inhibitor. Aromatase is an enzyme which effects aromatization of ring A in the metabolic formation of various steroid hormones. Various cancers, for example breast cancer, are dependent upon circulating steroid hormones which have an aromatic ring A. Such cancers can be treated by inhibition of the aromatization of the steroid ring A, and the compounds of the invention are useful for this purpose. It is used for the treatment of advanced breast cancer in postmenopausal women with disease progression following tamoxifen therapy and adjuvant therapy of postmenopausal women having early breast cancer.

The preparation of Anastrozole is described in EP 296749 started from 3,5-bis(bromomethyl)toluene (Ana-1) according to the following scheme:

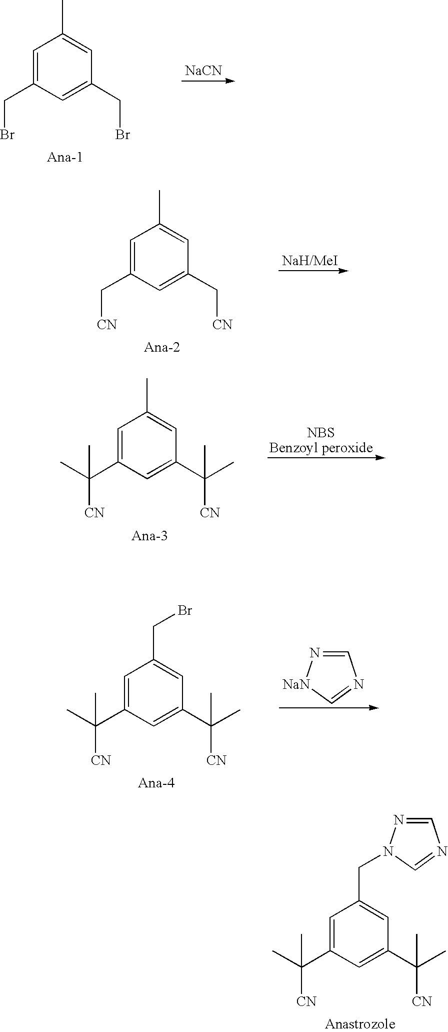

Reaction of Ana-1 with potassium cyanide or sodium cyanide in a mixed solvent of dichloromethane/water in the presence of tetrabutylammonium bromide at reflux temperature gave Ana-2. Treatment of Ana-2 with sodium hydride (60% dispersion in mineral oil), and iodomethane in dimethylformamide at room temperature for 2 hours gave 3,5-bis(2-cyanoisopropyl)toluene (Ana-3).

The Ana-3 was treated with N-bromosuccinimide (NBS) in carbon tetrachloride in the presence of benzoyl peroxide at reflux temperature to obtain the crude 3,5-bis (2-cyanoisopropyl)benzylbromide (Ana-4), which was then treated with 1,2,4-triazol sodium in dimethylformamide to obtained the crude Anastrozole. The crude Anastrozole was then purified first by flash column chromatography followed by crystallization from ethyl acetate/cyclohexane to obtain the purified product.

The Ana-3, 3,5-bis(2-cyanoisopropyl)toluene used as a starting material for the bromination reaction is commercially available.

The 3,5-bis(bromomethyl)toluene (Ana-1) mentioned above was not commercially available, and the method for the preparation of Ana-1 starting from 1,3,5-trimethylbenzene was found in the literature (J. Org. Chem., 2272, 1967)

Improved and commercially viable processes for the preparation of high purity Anastrozole by purification of Anastrozole via its isolated salt form are disclosed in WO 2005/105762 and US 20060035950. As compared to the processes described in EP 296749, the improved process also have the advantage of avoiding the usage of hazardous reagents, such as carbon tetra-chloride and benzoyl peroxide.

A novel process for the preparation of Anastrozole is disclosed in WO 2006/000836. The new process comprises two or more of the steps for preparing Anastrozole from 3,5-bis(2-cyanoisopropyl)toluene and the Anastrozole was prepared through novel intermediates.

A problem associated with the process described in EP 296749 is that the isomer of Anastrozole is an undesired product; thus requiring additional steps to separate it from Anastrozole. An improved process for preparing Anastrozole substantially free of undesired isomers is disclosed in US 20060189670.

WO 2006/108155 disclosed an alternative process for preparation and purification of Anastrozole. The new method for purification of the Anastrozole by selective extraction does not require the isolation of Anastrozole in a form of a salt, thus, the steps of transforming Anastrozole to its salt and then back to the free base, and the usage of toxic solvents, such as tetra-chloride, are avoided. Hence, this improved process can be adapted easily and efficiently to industrial scale. However, the reaction condition of Anastrozole synthesis from 3,5-bis(2-cyanoisopropyl)benzylbromide is limited to the temperature below 0° C., which is not a convenient operation. The long reaction time and the complicated steps, such as more than one temperature condition in one step, are also disadvantageous with respect to industrial application. Moreover, this method was found to be not successful in reducing the level of isomeric impurity of Anastrozole to 0.1%.

### SUMMARY OF THE INVENTION

The present invention provides a more efficient process for preparing Anastrozole of the following formula

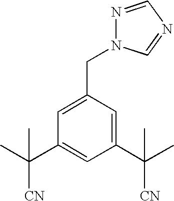

comprising reacting 3,5-bis(2-cyanoisopropyl)toluene with a brominating reagent in an organic solvent; and reacting with 1,2,4-triazole in an organic solvent at a temperature about 0 to 60° C. as the follow scheme.

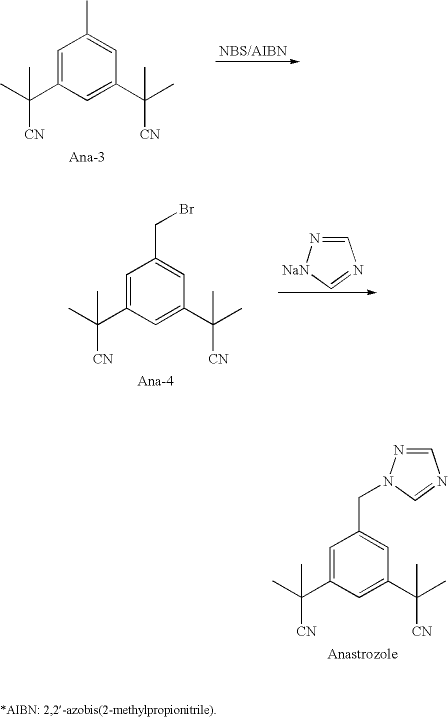

### BRIEF DESCRIPTION OF THE DRAWINGS

FIG. 1 shows the purity of 3,5-bis(2-cyanoisopropyl)benzylbromide (Ana-4) under different N-bromosuccinimide molar equivalent.

### DETAILED DESCRIPTION OF THE INVENTION

The present invention provides an improved, efficient process for preparing Anastrozole. Synthesis of Anastrozole without reducing the temperature below 0° C., easily operation, devoid of using carcinogenic reagent, and reducing the level of isomeric impurity of the Anastrozole to 0.1% are advantageous with respect to industrial application. Further, the present invention provides an improved process for preparing the 3,5-bis(bromomethyl)toluene from the 1,3,5-trimethylbenzene without the usage of toxic reagents, such as benzoyl peroxide and carbon tetra-chloride.

Accordingly, the present invention provides a process for preparing Anastrozole of formula VI

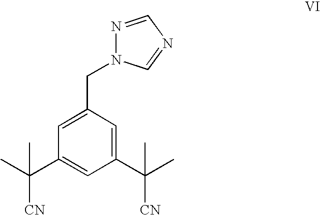

comprising the steps of:
(a) reacting 3,5-bis(2-cyanoisopropyl)toluene of formula IV

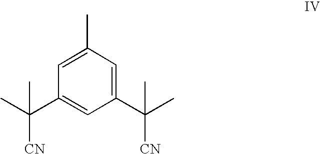

with a brominating reagent in an organic solvent to provide a compound of formula V

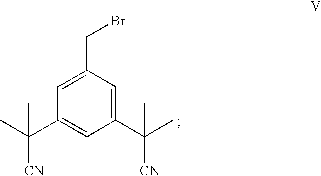

;and
(b) reacting the compound of formula V with 1,2,4-triazole in an organic solvent at a temperature about 0 to 60° C. as the follow scheme.

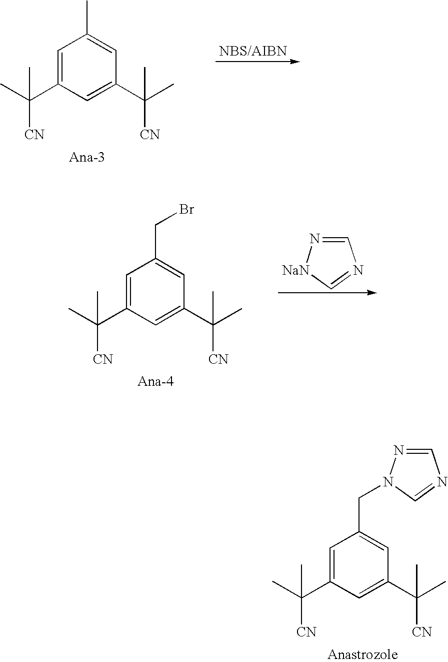

In the process of the present invention, the brominating reagent is not limited but selected from the group consisting of N-bromosuccinimide, 2,2′-azobis(2-methylpropionitrile), and benzoyl peroxide. In the preferred embodiment, the brominating reagent is N-bromosuccinimide and 2,2′-azobis(2-methylpropionitrile). The molar ratio of N-bromosuccinimide to the compound of formula-IV is 1.0 to 1.5. In the preferred embodiment, the molar ratio of N-bromosuccinimide to the compound of formula-IV is 1.0 to 1.2.

In the present invention, the organic solvent of step (a) is not limited but selected from the group consisting of acetonitrile, cyclohexane, dimethylformamide, methylisobutylketon, and carbon tetrachloride. Preferred organic solvent for reaction is acetonitrile. The reaction mixture is stirred and heated for about 0.5 to about 3 hours, preferably for about 1.5 to about 2 hours.

In the process of the present invention, the reaction of step (b) is made by the 1,2,4-triazole with molar equivalents in the range of 0.9 to 1.5. The organic solvent used in step (b) is not limited but selected from the group consisting of dimethylformamide, 2-propanol, methanol, n-heptane, and acetone. Preferred organic solvent for reaction is dimethylformamide. The mixture is stirred at the temperature in the range of 0 to 60° C. for 0.5 to 4 hours, preferably in the rage of 5 to 35° C. After completion, the crude Anastrozole is further purified. The crude Anastrozole is purified by column and eluted with ethyl acetate, and followed by crystallization from organic solvent. The crystallization is made by the following steps:
(a) reacting the purified Anastrozole with an organic solvent to form a mixture;
(b) heating the mixture of step (a) at the temperature of 65° C. to reflux; and
(c) cooling to a temperature of about 0 to 15° C.

The organic solvent of crystallization is not limited but selected from the group consisting of 2-propanol, ethyl acetate, and cyclohexane. In the preferred embodiment, the organic solvent is 2-propanol. The process of the present invention provides the final produce with less than 0.1% impurities.

The examples below are non-limited and are merely representative of various aspects and features of the present invention.

### EXAMPLE

### Example 1

### Preparation (2-cyanoisopropyl)benzylbromide (Ana-4)

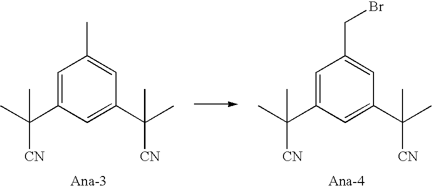

General procedures for the preparation of crude Ana-4:
1. To a flask were added Ana-3, suitable amount of N-bromosuccinimide (NBS), around 0.015 meq of initiator, and an organic solvent.
2. The mixture was stirred and heated at reflex.
3. The reaction progress was monitored by HPLC.
4. After completion, the solvent was evaporated under reduced pressure.
5. To the residue was added suitable amount of ethyl acetate and water.
6. The mixture was stirred for about 10 minutes and then phase separated.
7. The organic layer was washed with water three times.
8. The organic layer was concentrated to dryness to obtain crude Ana-4.

Four major parameters were selected including: 1) selection of initiator, 2) selection of solvent, 3) molar ratio of N-bromosuccinimide toward Ana-3, and 4) reaction time.

1.1 Selection of Initiator

By following the general procedure (cyclohexane as organic solvent, 1.2 meq of N-bromosuccinimide was used, reaction temperature: at reflux, reaction time: 1˜2 hours), two commercially available peroxide such as benzoyl peroxide and 2,2′-azobis(2-methylpropionitrile) (AIBN) were selected, and the results were listed as shown in Table 1.

TABLE 1

| | | LC purity (%) | | |
|---|---|---|---|---|
| | | Ana-3 | Ana-4 | Side-product |
| Bat. No. | initiator | RRT = 0.93 | RRT = 1.00 | RRT = 1.50 |
| 163R076 | AIBN | 29.7 | 64.4 | 3.76 |
| 163R085 | Benzoyl | 30.7 | 59.9 | 5.78 |
| | peroxide | | | |

Based on the results, it was concluded that the same impurity profiles were obtained while the use of different initiators. Surprisingly, the use of 2,2′-azobis(2-methylpropionitrile) (AIBN) has a better conversion than that of benzoyl peroxide.

1.2 Selection of Solvent

By following the general procedure (1.2 meq of NBS and 0.015 meq of AIBN was used), various solvents, such as cyclohexane (CH), dimethylformamide (DMF), methylisobutylketon (MIBK), CCl₄, and acetonitrile were selected for our study, and the results were listed as shown in Table 2.

TABLE 2

| | | | | LC purity (%) | | |
|---|---|---|---|---|---|---|
| | | Reaction | Reaction | Ana-3 | Ana-4 | Side-product |
| Bat. No. | solvent | time | temperature | RRT = 0.93 | RRT = 1.00 | RRT = 1.50 |
| 163R076 | CH | 1 hr | Reflux | 29.7 | 64.4 | 3.76 |
| 163R082 | DMF | 4 hrs | 80° C. | 94.7 | 3.53 | — |
| 163R103 | MIBK | 1.5 hrs | Reflux | 65.9 | 21.8 | 0.17 |
| 63R109-A | CCl₄ | 2 hrs | Reflux | 2.87 | 70.0 | 27.1 |
| 63R109-B | CH₃CN | 2 hrs | Reflux | 1.14 | 81.0 | 17.6 |

Based on the results, it was concluded that the use of acetonitrile obtained the best chemical conversion for the preparation of Ana-4.

1.3 Molar Ratio of NBS Toward Ana-3

By following the general procedures (acetonitrile as solvent, 0.015 meq of AIBN was used, and heated at reflux for certain period of time), various molar ratio of NBS toward Ana-3 were tested, and the results were listed as shown in Table 3.

TABLE 3

| | | | LC purity (%) | | |
|---|---|---|---|---|---|
| | NBS | Reaction | Ana-3 | Ana-4 | Side-product |
| Bat. No. | (meq) | time (hr) | RRT = 0.93 | RRT = 1.00 | RRT = 1.50 |
| 163R0128 | 1 | 1.5 | 5.15 | 84.8 | 9.98 |
| 163R112 | 1.05 | 3 | 4.40 | 85.5 | 10.0 |
| 163R126 | 1.1 | 3 | 2.43 | 83.05 | 14.5 |
| 163R109- | 1.2 | 2 | 1.14 | 81.0 | 17.6 |
| B | | | | | |
| 163R121- | 1.5 | 2 | 0.20 | 69.5 | 28.6 |
| B | | | | | |
| 163R138 | 1.05 | 2 | 3.74 | 83.9 | 12.3 |

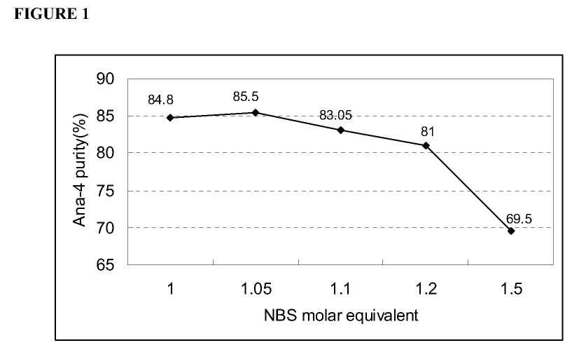

Above data is shown as FIG. 1. Based on the results, it concluded that the molar equivalent of NBS used for the reaction should be in the range of 1.0˜1.2 to obtain the crude Ana-4 of purity more than 80%.

1.4 Reaction Time

By following the general procedures (acetonitrile as solvent, 1˜1.1 meq of NBS and 0.015 meq of AIBN was used, and heated at reflux for certain period of time), the reaction progress was monitored at the specified time period, and the results obtained were listed as shown in Table 4.

TABLE 4

| | | | LC purity (%) | | | |
|---|---|---|---|---|---|---|
| | NBS | Reaction | Ana-3 | Ana-4 | Side-product | Crude yield |
| Bat. No. | (meq) | time (hr) | RRT = 0.93 | RRT = 1.00 | RRT = 1.50 | (%) |
| 163R112 | 1.05 | 2 | 4.61 | 84.1 | 11.1 | 96.0 |
| | | 3 | 4.4 | 85.2 | 10.0 | |
| 163R126 | 1.1 | 1 | 2.46 | 82.4 | 14.6 | 101.5 |
| | | 1.5 | 2.35 | 82.2 | 14.6 | |
| | | 2 | 2.38 | 82.6 | 14.1 | |
| | | 2.5 | 2.45 | 82.3 | 14.5 | |
| | | 3 | 2.43 | 83.0 | 14.5 | |
| 163R128 | 1 | 0.5 | 2.4 | 83.0 | 14.4 | 90.4 |
| | | 1 | 5.12 | 84.6 | 10.1 | |
| | | 1.5 | 5.15 | 84.8 | 9.98 | |
| 163R138 | 1.05 | 1 | 3.63 | 84.4 | 11.9 | 100.5 |
| | | 1.5 | 3.68 | 84.2 | 12.0 | |
| | | 2 | 3.74 | 83.9 | 12.3 | |
| 163R168 | 1.05 | 1 | 3.68 | 83.9 | 12.4 | 101 |
| | | 1.5 | 3.47 | 84.5 | 11.8 | |
| | | 2 | 3.54 | 84.5 | 11.9 | |

Based on the results, it was concluded that the reaction time for the preparation of Ana-4 from Ana-3 could be in the range of 0.5˜3 hours to obtain the crude Ana-4 of purity more than 80%, and the crude yield of more than 90%.

The crude Ana-4 was used directly for the preparation of Anastrozole without further purification.

### Example 2

### Preparation of Anastrozole

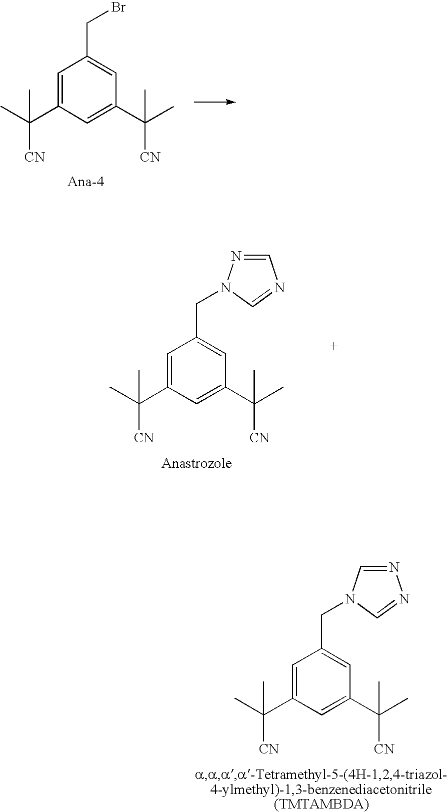

Anastrozole was prepared by the reaction of crude Ana-4 with 1,2,4-triazole sodium (˜2.7 molar equivalent) in dimethylformamide (DMF) at room temperature.

Four major parameters were selected including: 1) solvent 2) the reaction temperature 3) molar ratio of 1,2,4-triazole sodium toward Ana-4, and 4) the reaction time.

A potential impurity named α,α,α′,α′-tetramethyl-5-(4H-1,2,4-triazol-4-ylmethyl)-1,3-benzenediacetonitrile (TMTAMBDA, structure as shown in the above scheme) caused by side-reaction was identified.

General procedures for the preparation of crude Anastrozole:
1. To a flask was added Ana-4, suitable amount of 1,2,4-triazole sodium, and an organic solvent.
2. The mixture was stirred.
3. The reaction progress was monitored by HPLC.
4. After completion, ethyl acetate (10V) and water (10V) was added.
5. The mixture was stirred for about 10 minutes and then phase separated.
6. To the aqueous layer was added ethyl acetate (5V).
7. The mixture was stirred for about 10 minutes and then phase separated.
8. The aqueous layer was discarded, and the organic layers were combined.
9. The combined organic layer was washed with water (10V) four times.
10. The organic layer was concentrated to dryness to obtain the crude Anastrozole.

2.1 Selection of Solvent

By following the general procedures (1 meq of 1,2,4-triazole.sodium was used), various solvents, such as DMF, 2-propanol (IPA), MeOH, n-heptane, acetone were selected, and the results were listed as shown in Table 5.

TABLE 5

| | | | LC purity (%) | | | | TMTAMBDA/ |
|---|---|---|---|---|---|---|---|
| | | Temp. | TMTAMBDA | Anastrozole | Ana-3 | Side-product | Anastrozole |
| Bat. No. | solvent | (° C.) | RT = 3.1 | RT = 4.2 | RT = 15 | RT = 24.4 | ratio |
| 173R063 | DMF | 10~25 | 5.88 | 67.0 | 5.95 | 20.1 | 1/11.4 |
| 173R042A | IPA | Reflux | 11.4 | 66.1 | 6.78 | 14.9 | 1/5.8 |
| 173R042B | MeOH | Reflux | 10.6 | 64.5 | 6.78 | 16.0 | 1/6.1 |
| 173R042C | n-Hep. | Reflux | 6.75 | 46.9 | 22.1 | 21.2 | 1/6.9 |
| 173R042D | Acetone | Reflux | 8.92 | 67.8 | 6.83 | 15.7 | 1/7.6 |

Based on the above results, the chemical conversion from Ana-4 to Anastrozole was around 67% when DMF, IPA, and Acetone were selected as the solvent.

However, when the ratio of TMTAMBDA/Anastrozole was considered, the best result was obtained (which means the ease of purification) while the use of DMF.

2.2 The Reaction Temperature

By following the general procedures (DMF as the solvent, 0.9 meq of 1,2,4-triazole.sodium was used), the reaction temperature was studied and the results were listed as shown in Table 6.

TABLE 6

| | | LC purity (%) | | |
|---|---|---|---|---|
| | | TMTAMBDA | Anastrozole | Crude yield |
| Bat. No. | Temp. (° C.) | RRT = 0.75 | RRT = 1.00 | (%) |
| 163R182-1 | 15~25 | 5.36 | 67.9 | 104.7 |
| 163R187-1 | 5~15 | 5.11 | 68.5 | 103.4 |
| 163R190-1 | 25~35 | 5.53 | 65.1 | 106.2 |

Based on the above results, it was concluded that the reaction temperature for the preparation of Anastrozole could be in the range of 5˜35° C., and the yield of the crude was around 100%.

2.3 Molar Ratio of 1,2,4-Triazol.Sodium Toward Ana-4 and the Reaction Time.

By following the general procedures, experiments were performed and the results obtained were listed as shown in Table 7.

TABLE 7

| | 1,2,4-triazol | | LC purity (%) | | | Crude |
|---|---|---|---|---|---|---|
| | sodium | Reaction | TMTAMBDA | Anastrozole | Ana-4 | yield |
| Bat. No. | (meq) | time (hr) | RRT = 0.75 | RRT = 1.00 | RRT = 3.89 | (%) |
| 163R114-B | 1.5 | 4 | 5.48 | 75.8 | ND | NA |
| 163R131 | 1.2 | 1 | 4.8 | 60.8 | ND | NA |
| 163R134 | 1.0 | 0.5 | 5.22 | 70.7 | ND | NA |
| | | 1 | 5.34 | 70.6 | ND | |
| 163R140 | 1.0 | 0.5 | 5.46 | 66.9 | ND | 115 |
| | | 1 | 4.88 | 67.6 | ND | |
| 163R184 | 0.9 | 0.5 | 4.80 | 67.8 | ND | 104.7 |
| | | 1 | 5.13 | 67.2 | ND | |
| 163R186 | 0.9 | 0.5 | 4.51 | 67.7 | ND | 103.4 |
| | | 1 | 4.46 | 67.9 | ND | |
| 163R189 | 0.9 | 0.5 | 4.91 | 65.31 | ND | 106.2 |
| | | 1 | 5.12 | 64.7 | ND | |

Based on the results, it was concluded that 1) the use of 0.9˜1.5 molar equivalent of 1,2,4-triazol sodium was plenty to push the reaction to complete, 2) the reaction time could be in the range of 0.5˜4 hours, 3) the purity of the crude could be more than 60%, and 4) the yield of the crude was around 100%.

### Example 3

### Purification of Crude Anastrozole

The crude Anastrozole was column purified (eluting with ethyl acetate), and fractional collected. The results obtained were listed as shown in Table 8.

TABLE 8

| | Crude purity | | Anastrozole | |
|---|---|---|---|---|
| Bat. No. | (%) | Fraction | Purity (%) | Yield (%) |
| 163R141 | 67.6 | 1 | 93.4 | 17.3 |
| | | 2 | 98.9 | 19.4 |
| | | 3 | 98.1 | 15.6 |
| 173R014-1 | 68.5 | 4–14 | 99.1 | 53.2 |
| 173R094-2 | 65.6 | 2 | 99.6 | 66.1 |

Based on the results, it was concluded that 1) the crude could be easily purified by column chromatography to obtain the purified product of purity more than 98%, and 2) the yield for the purification was around 50˜66%.

### Example 4

### Crystallization of Anastrozole

It was found that one part of Anastrozole could be dissolved in a hot solution of 1.5 parts (by volume) of ethyl acetate and 3 parts (by volume) of cyclohexane, and then crystallized at around 50° C.

Another organic solvent (2-propanol) was found feasible for crystallization, one part of Anastrozole could be dissolved in a hot solution of 2.5 parts (by volume) of 2-propanol, and then crystallized at around 20° C.

Therefore, the crystallization procedures were performed as following:
1. To a flask was added purified Anastrozole, ethyl acetate (1.5V), and cyclohexane (3V) or 2-propanol (2.5V).
2. The mixture was stirred and heated at 65° C. to reflux to obtain a solution.
3. The solution was cooled slowly to about 15° C., and kept at the temperature for about an hour.
4. The crystals formed were filtered to obtain Anastrozole wet cake.
5. The wet cake was dried under vacuum (˜15 torr) at around 40˜50° C. for about 6 hours to obtain the final product.

By following the procedures, Anastrozole was crystallized, and the results obtained were listed as shown in Table 9.

TABLE 9

| | | Purity before | Purity after | |
|---|---|---|---|---|
| | Crystallization | crystallization | crystallization | Crystallization yield |
| Bat. No. | solvent | (%) | (%) | (%) |
| 163R142-2 | EA/cyclohexane | ~98.5 | 99.7 | 91.6 |
| 173R068-3 | 2-propanol | 99.3 | 99.9 | 91.8 |
| 173R088-2 | 2-propanol | 99.7 | 99.9 | 91.0 |
| 173R090-2 | 2-propanol | 99.5 | 99.9 | 89.6 |
| 173R098-2 | 2-propanol | 99.5 | 99.9 | 90.6 |

Based on the results, it was concluded that 1) Anastrozole can be easily crystallized from a mixed solvent of ethyl acetate/cyclohexane or 2-propanol to obtain a purity of more than 99.5%, and 2) the yield for the crystallization step was around 90%.

Cyclohexane was a class 2 solvent, and EA and 2-Propanol were class 3. For safety reason, 2-Propanol was selected as the solvent for the crystallization of Anastrozole.

### Example 5

### Optimal Procedures for the Preparation of Anastrozole from Ana-4

1. To a flask was added Ana-4, suitable amount of 1,2,4-triazole sodium, and DMF(5V).
2. The mixture was stirred at 5˜35° C. for 1.5 hr.
3. The reaction progress was monitored by HPLC.
4. After completion, ethyl acetate (10V) and water (10V) was added.
5. The mixture was stirred for about 10 minutes and then phase separated.
6. To the aqueous layer was added ethyl acetate (5V).
7. The mixture was stirred for about 10 minutes and then phase separated.
8. The aqueous layer was discarded, and the organic layers were combined.
9. The combined organic layer was washed with water (10V) four times.
10. The organic layer was concentrated to dryness to obtain the crude Anastrozole.
11. The crude Anastrozole was column purified (eluting with ethyl acetate), and fractional collected.
12. The fraction collected was then crystallized from 2-propanol (2.5V) to obtain the purified Anastrozole.

By following the optimal procedures, the preparation of Anastrozole was repeated, and the results obtained were listed as shown in Table 10.

TABLE 10

| | | | Column | Column | | |
|---|---|---|---|---|---|---|
| | | Crude | purified | purified | | |
| | Crude | purity | Yield | purity | Crystallization | Crystallization |
| Bat. No. | yield (%) | (%) | (%) | (%) | yield (%) | purity (%) |
| 173R068-3 | 99.0 | 67.1 | 69.1 | 99.3 | 91.8 | 99.9 |

Conclusion:

Based on the above results, it was concluded that by following the optimal procedures for the preparation of Anastrozole from Ana-4, 1) the crude yield was around 100%, and the crude purity was around 65%, 2) the yield after chromatography was around 65%, and the purity increased to around 99%, and 3) the crystallization yield was around 90% and the purity of Anastrozole increased to about 99.9%.

Based on the above results, the level of isomeric impurity of Anastrozole is reduced to 0.1%.

What is claimed is:

1. A process for preparing Anastrozole of formula VI 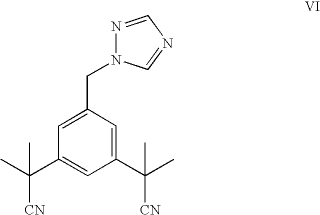
comprising the steps of:
(a) reacting 3,5-bis(2-cyanoisopropyl)toluene of formula IV 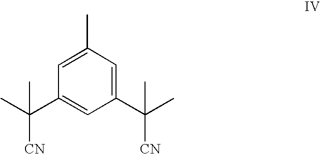
with a brominating reagent in an organic solvent to provide a compound of formula V 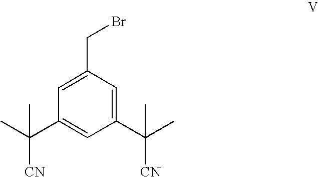
;and
(b) reacting the compound of formula V with 1,2,4-triazole in an organic solvent at a temperature about 0° C. to 60° C.

2. The process according to claim 1, wherein the brominating reagent is selected from the group consisting of N-bromosuccinimide, 2,2′-azobis(2-methylpropionitrile), and benzoyl peroxide.

3. The process according to claim 2, wherein the brominating reagent is N-bromosuccinimide and 2,2′-azobis(2-methylpropionitrile).

4. The process according to claim 1, wherein the organic solvent of step (a) is selected from the group consisting of acetonitrile, cyclohexane, dimethylformamide, methylisobutylketon, and carbon tetrachloride.

5. The process according to claim 4, wherein the organic solvent is acetonitrile.

6. The process according to claim 1, wherein the reaction mixture of step (a) is further heated for about 0.5 to about 3 hours.

7. The process according to claim 1, wherein the molar ratio of N-bromosuccinimide to the compound of formula-IV is 1.0 to 1.5.

8. The process according to claim 7, wherein the molar ratio of N-bromosuccinimide to the compound of formula-IV is 1.0 to 1.2.

9. The process according to claim 1, wherein the organic solvent used in step (b) is selected from the group consisting of dimethylformamide, 2-propanol, methanol, n-heptane, and acetone.

10. The process according to claim 9, wherein the organic solvent used in step (b) is dimethylformamide.

11. The process according to claim 1, wherein the reaction temperature of step (b) is in the range of 5 to 35° C.

12. The process according to claim 1, wherein the mixture reaction of step (b) is maintained in the range of 0.5 to 4 hours.

13. The process according to claim 1, wherein the reaction of step (b) is made by the 1,2,4-triazole with molar equivalents in the range of 0.9 to 1.5.

14. The process according to claim 1, further comprising purification of crude Anastrozole by chromatography and crystallization of purified product.

15. The process according to claim 14, wherein the crude Anastrozole is purified by column and eluted with ethyl acetate.

16. The process according to claim 14, wherein the crystallization is from organic solvent.

17. The process according to claim 14, wherein the crystallization is made by the following steps:
(a) reacting the purified Anastrozole with an organic solvent to form a mixture;
(b) heating the mixture of step (a); and
(c) cooling to a temperature of about 0 to 15° C.

18. The process according to claim 14 which has a content of impurities is less than 0.1%.

19. The process according to claim 17, wherein the organic solvent is 2-propanol, ethyl acetate, and cyclohexane.

20. The process according to claim 17, wherein the temperature of step (b) is in a rage of 65° C. to reflux.
